(19) United States
(12) Patent Application Publication
(10) Pub. No.: US 2015/0125824 A1
(43) Pub. Date: May 7, 2015

(54) Application system for facile 3-dimensional application of medical, cosmetic or drug-containing dental care products

(71) Applicant: Heraeus Kulzer GmbH, Hanau (DE)

(72) Inventors: Susanne BUSCH, Neu Anspach (DE); Andreas UTTERODT, Neu Anspach (DE); Michael GERLACH, Hofheim (DE); Ralf SUFFEL, Neu Anspach (DE)

(21) Appl. No.: 14/481,268

(22) Filed: Sep. 9, 2014

(30) Foreign Application Priority Data
Sep. 9, 2013 (DE) 10-2013 109 848.7

(51) Int. Cl. A61C 19/06; A61K 6/00

(52) U.S. Cl. CPC A61C 19/063; A61K 6/0017
USPC 433/217.1; 264/16

(57) ABSTRACT

The invention discloses a partially elastic shaped body in the form of a negative image of a dental arch containing a matrix based on a gel containing an active substance or a system with a mineralizing effect as well as an application system for the treatment of teeth. The application system comprises an application dental splint that is well-suited for use on the upper and/or lower jaw, whereby the dental splint can accommodate at least one first three-dimensional shaped body that is adapted to the splint and contains a matrix, preferably the dental splint can be deformed by heat. The matrix of the at least one first and optionally further shaped body contains at least one active substance or a system with a mineralizing effect containing phosphates, calcium, and fluorides. The method for producing the application system and a kit comprising the application system are also a subject matter of the invention. According to the invention, the application system and kit are used for depositing apatite, in particular needle-shaped fluorapatite crystals. According to the invention, it is feasible to deposit more than or equal to 1 μm apatite, in particular fluorapatite, on tooth surfaces over one sleeping period or during the day in order to seal or brighten porous tooth surfaces.

This application claims priority of German Patent Application No. 10 2013 109 848.7, filed on Sep. 9, 2013.

The invention discloses a partially elastic, arch-shaped shaped body in the form of a negative image of a dental arch containing a matrix based on a gel containing an active substance or a system with a mineralizing effect as well as an application system for the treatment of teeth. The application system comprises an application dental splint that is well-suited for use on the upper and/or lower jaw, whereby the dental splint can accommodate at least one first three-dimensional shaped body that is adapted to the splint and contains a matrix, preferably the dental splint can be deformed by heat. The matrix of the at least one first and optionally further shaped body contains at least one active substance or a system with a mineralizing effect containing phosphates, calcium, and fluorides. The method for producing the application system and a kit comprising the application system are also a subject matter of the invention. According to the invention, the application system and kit are used for depositing apatite, in particular needle-shaped fluorapatite crystals. According to the invention, it is feasible to deposit more than or equal to 1 μm apatite, in particular fluorapatite, on tooth surfaces over one sleeping period or during the day in order to seal or brighten porous tooth surfaces.

Teeth are hard biomaterials in the form of composites based on proteins and apatite comprising calcium and phosphate. Enamel, i.e. the outer layer of the crown of the tooth, is the hardest part of the tooth and contains no viable cells. It consists of inorganic crystals which typically are present in highly oriented arrangements. Enamel is a tissue, which, once it is produced, remains nearly unchanged for life since the cells involved in building up the teeth die as soon as tooth formation is completed. Finished enamel consists of approx. 95% by weight apatite, approx. 3% by weight proteins and lipids, and approx. 2% by weight water.

In order to prevent or repair tooth damage, in particular due to caries, attempts to use remineralizing systems have been made for a long time. These initially involved the application of calcium phosphate compounds to improve the properties of the teeth.

Such one-component systems attempting to apply pre-made tooth substance, for example apatite, hydroxyapatite or other calcium phosphate compounds, to the teeth, are described, inter alia, in EP 0666730 B1 or WO 01/95863. It is a problem of said systems that treating the tooth substance with calcium phosphate compounds does not lead to the growth of an apatite that is structurally similar to the tooth substance, but rather to mere deposition of apatite crystals on the tooth substance, whereby the morphology of the apatite crystals is very different from that of tooth substance. Accordingly, there is no strengthening effect on the enamel and no permanent filling of lesions, since the deposited apatite crystals do not comprise sufficient similarity and adhesion to the tooth substance.

Due to modern dietary habits, which often involve acidic food items, erosions of the dental hard substance that are not due to bacteria are on the rise [Dentale Erosionen: Von der Diagnose zur Therapie, Adrian Lussi, Thomas Jaeggi, Quintessenz Verlag].

But not only food items such as strongly acidified sweets, soft drinks or alcopops play a role in this regard, but the trend towards nutrition containing more fruit can also lead to dental problems. The continuous exposure to acids makes the enamel thinner and more porous. In extreme cases, the enamel can be dissolved totally and/or abraded such that the sensitive dentine is exposed. In the neck region of the teeth, in particular, which is protected by a very thin layer of enamel only, this occurs frequently. Acid-caused erosion can then proceed at even faster rates since dentine is more acid-soluble than enamel and wedge-shaped defects in the dental hard substance are often caused. Exposed dentine leads to sensitive, pain-sensitive teeth. However, sensitive dental necks can just as well be a consequence of inappropriate brushing habits. Increasing age is another reason for the enamel getting thinner. Habitual bruxism can also abrade the enamel at the incisal edges. Due to the improved prophylaxis in dentistry and strict addition of fluoride to most toothpastes and additional care products, caries is decreasing, but since the population in the industrialised countries is ageing and functional teeth have to work longer, the significance of non-cariogenic losses of dental hard substance is increasing as well.

Some forms of administration described to be suited for inducing the mineralization of apatite on the surface of teeth are known. U.S. Pat. No. 6,521,251 describes a composition that contains not only carbamide peroxide, but also calcium phosphates which are slightly more soluble than apatite, such as mono-, di- or tricalcium phosphate. But still, all these calcium phosphates are poorly water-soluble, such that the tooth cleaning means described are expected to have an abrasive rather than a remineralizing effect. In fact, U.S. Pat. No. 5,851,514 describes, inter alia, the addition of dicalcium phosphate as an abrasive.

U.S. Pat. No. 6,419,905 mentions the addition of potassium salts (e.g. citrate) and fluoride to the peroxide. Fluoride is suited for binding, in particular, calcium and phosphate ions from the saliva, leading to the precipitation of fluorapatite. If no other ions are added, the formation of CaF₂ has also been observed. Calcium fluoride particles can be stored in the plaque and can release fluoride for extended periods of time since they are more soluble than the apatite of the hard dental substance. However, conscientious repeated daily cleaning of the teeth largely removes the plaque. Accordingly, the effect of calcium fluoride is short-lived and the fluoride-containing products need to be applied in regular intervals. No formation of new apatite has been observed with products of this type.

Patent JP20000051804 describes the concurrent use of concentrated phosphoric acid, conc. H₂O₂, and fluorapatite powder. The use of concentrated phosphoric acid in this context appears questionable as this substance can dissolve healthy enamel to a notable degree. Moreover, the bleaching solution is strongly irritating and must not contact the gingiva, although this is true, at a lesser level, of all tooth-bleaching agents having an oxidative effect. Moreover, repeated application does not lead to the build-up of a mineralization layer.

An acid-free application is described in US 20050281759. Calcium peroxophosphate is proposed as essential ingredient in this context. The underlying rationale being that a single substance is to have the brightening and remineralizing effect, since the release of calcium and phosphate ions is triggered parallel to the oxidation. It is not clear whether or not the salts can attain any significant build-up of apatite during their relatively short period of action. U.S. Pat. No. 6,303,104 describes an oxidant-free two-component system consisting of soluble calcium and phosphate salts, which is claimed to have a brightening effect as well. The brightening is said to be caused through the addition of sodium gluconate, which forms complexes with staining metal ions (e.g. iron) from the enamel. Mixing of the components is expected to immediately lead to precipitation of the poorly-soluble calcium phosphates and it is not obvious why there should be pronounced remineralization, even more so as the product is a toothpaste to which the tooth surfaces is exposed for no more than a few minutes at a time. U.S. Pat. No. 6,102,050 describes a dental floss having titanium dioxide particles that is said to have a brightening, remineralizing, and desensitising effect on the interdental surfaces. Titanium dioxide microparticles of a size of 0.1 to 1.5 μm are to act both as a mild abrasive and are to be absorbed by the enamel, which is said to be associated with a brightening effect. Presumably, the particles can no more than get incorporated mechanically into suitable hollow spaces which does not promise to lead to stable anchoring and can have no more than a temporary effect.

All patents described thus far fail to take into consideration that bio-minerals attain their high degree of structural organization and stability only because they are formed in the presence of specific biomolecules that define the formation of the micro- and macro-structure.

WO 2005/027863 describes a tooth care product that is said to possess a cleaning, remineralizing, desensitizing, and brightening effect. The nano-scale apatite-gelatine composite proposed as active component for remineralization and brightening precipitates in the presence of an aqueous gelatine solution and thus has polypeptides incorporated into it. This material is said to form a protective layer of dentine-like structure on the surface of the tooth due to so-called “neo-mineralization”, whereby the protective film is said to smoothen the surface and to be able to seal open dentine tubules. This effect is not comprehensible for a toothpaste, since said tooth care product preferably contains only 0.01-2% by weight “nanite” (WO 01/01930). The active substances can act for no more than a few minutes daily. Any significant or surface-covering deposition of mineral is doubtful. Moreover, no deposition of mineral on enamel is described. No continuous increase in the thickness of the film upon extended application of the care product is described either. Moreover, the porous, poorly ordered structure of dentine is not capable of protecting the tooth from corrosive attacks. The commercially available product, Tooth Mousse or Mi-Paste, is based on patent specifications by Reynolds [WO 98/40406] and is said to remineralize porous enamel. The invention is based on casein (CPP) having a stabilizing effect on amorphous calcium phosphate (ACP). In contact with the hard dental substance, the CPP-ACP agent is to remineralize into hydroxylapatite. A protective film of dentine-like structure of this type appears unsuited to provide long-term protection.

It is common to all patents that they only refer to remineralization without documenting same and/or without having a desensitizing effect. US 2012/0027829A1 describes the formation of hydroxylapatite layers (HAP) on dentine by repeatedly applying pasty mixtures of propylene glycol, glycerol, xylitol, polyethylene glycol, cetylpyridiniumchloride, tetracalcium phosphate, and an alkali salt of phosphoric acid to the teeth. Since tetracalcium phosphate reacts immediately with phosphoric acid salts in the presence of water, two separate pastes are produced first and mixed only right before application. No formation of HAP on enamel is described and no data is provided on the layer formed, which also was not reproducible in own experiments.

The technique described in US2005220724 and DE 10 2004 054 584.7 provides a fluorapatite layer which possesses enamel-like strength and increases in thickness upon repeated application. Water-soluble phosphate and fluoride salts are incorporated in the buffered gel A, whereas calcium ions are incorporated in gel B. Optionally separated through an ion-free protective layer, the gelatine gels, which are solid at physiological temperature, are applied one after the other, while heating, to in-vitro tooth surfaces. An increase of the thickness of the layer as a function of the exchange cycles of the gels can be observed. The growth rates are 0.5 to 5.0 μm/day. The biological structures of the tooth substance are replicated individually by the fluorapatite. Hollow spaces formed by exposed dentine tubuli are closed after a number of exchange cycles.

Regarding the use in humans, it is inconvenient that the gels need to be heated before application. The application of the second and third gel layer may cause underlying, previously applied gel layers to liquefy again and mix with the upper layers in undesirable manner. Small amounts applied as described, in particular, dry out quickly upon exposure to air and are then difficult to liquefy by heating them. The method hardly allows exactly defined amounts of gel of even thickness to be applied to the tooth. Moreover, the three gel layers, each being up to 6 mm in thickness, are quite bulky, which leads to problems in the case of protective systems, such as splints or plasters, as space for large gel reservoirs needs to be created in this case.

Moreover, the method becomes increasingly elaborate when the entire jaw including all tooth surfaces is to be treated. Since an application period for the formation of fluorapatite should not be less than 8 hours under ideal conditions, it would be of advantage if the patient could use the system himself/herself by using it before going to bed. For this, the patient would have to warm up the gels and place them precisely on the teeth, which is very difficult since warmed-up liquid gelatine is very tacky. Moreover, the lips interfere with the application of the liquid gels. Moreover, this is associated with a major risk of burns. It is also disadvantageous that the gels stay liquid and do not safely adhere on the teeth in the oral environment.

Since the gels leak despite the presence of protection, such as, e.g., an individualized deep-drawing splint, the splint needs to be sealed with a suitable sealing system, which renders the method even more complicated.

The use of pre-made gel strips in DE 102006055223 A1 is advantageous in that there is no cumbersome heating involved and the strips are of the same thickness. However, one major disadvantage is that the strips reach only partial regions of the teeth. However, since erosions basically affect all surfaces of teeth, it would be desirable to have the mineralization kit exert its effect in all places. Moreover, it is very cumbersome to unpack the two strips and to insert them, for example, into a deep-drawing splint, which, in addition, also needs to have a reservoir for the gel strips. Moreover, there is a major risk that the gels are applied to the tooth in the wrong order, which renders the system ineffective, or that the strips are not placed one on the other flush or perfectly fitting, which might reduce the effect. The problem of sealing is not solved satisfactorily either. Since the strips liquefy at the conditions prevailing in the oral environment, sealing is required though in order to prevent the active substances from leaking into the oral space.

It was the object of the invention to provide formulations and systems that enable the application of active substances, compounds or solid deposited substances, in particular of crystalline solids, and need direct and prolonged contact to the surface of the tooth for this purpose. Preferably, the application or deposition should be simplified and designed more efficiently. Moreover, the invention was to render it feasible to reach virtually all tooth surfaces of an arch of the jaw, in particular of a dental arch of a lower or upper jaw, without the products potentially leaking into the oral cavity. Using the system, it should preferably and alternatively be possible to apply a wide variety of dental care products, dental cosmetics or dental devices that should exert their effect everywhere on the teeth. Moreover, the deposition of apatite on the tooth surfaces should proceed more pleasantly and more efficiently. Furthermore, the deposition on the tooth surfaces should be made more homogeneous and preferably the possibility of confusing mineralization matrices should be prevented.

The object is solved through a three-dimensional shaped body (form body) having a matrix, whose matrix remains solid at the conditions prevailing in the mouth and is enriched in active substances. The active substances can be released to the teeth in delayed manner over a period of time. In this context, the three-dimensional shaped body, and thus the three-dimensional matrix, takes the shape of the negative image of a simplified row of teeth or part of a row of teeth. Organic gel-forming agents, such as Agar-Agar, carboxymethylcellulose, polyacrylic acid, silicone elastomers, hydrogels, denatured collagen (gelatine) or mixtures thereof, possibly made poorly soluble at the conditions prevailing in the mouth through chemical fixation, can be used as matrix.

The objects are solved through a formulation, an application dental splint, and a kit as described hereinbelow as well as through the methods for producing the formulation as also described hereinbelow.

Accordingly, the subject matter of the invention is at least one partially elastic, arch-shaped, three-dimensional shaped body comprising a matrix made of gel, whereby the shaped body is selected from a) a first three-dimensional shaped body containing a matrix comprising at least one gel, and b) a further three-dimensional shaped body containing a second matrix comprising at least one gel, and the at least one matrix, in particular the one and/or the second matrix, each independently contains at least one active substance, one pharmacologically tolerable salt, solvat of an active substance or a system having a mineralizing effect.

Whereby each shaped body independently corresponds, at least in part, to a negative image of a dental arch and can be applied in essentially form-fitting or perfectly fitting manner to at least one tooth or at least partially to a dental arch, or the shaped body is situated at a distance from at least one tooth of a dental arch and each shaped body independently comprises reduced solubility with respect to aqueous media, at least in part, in at least one plane as compared to the matrix, whereby the plane, in particular planes, or at least a partial envelope (casing) serves as membrane.

### BRIEF DESCRIPTION OF THE DRAWINGS

The invention will now be described in greater detail with reference to the drawings, wherein:

FIGS. 1a and 1b depict a CAD model of the tool for producing the two mineralization components in the form of a splint (first or further shaped body).

FIG. 2 shows the three components: 1. Protective splint (application dental splint), 2. Calcium component (first shaped body), 3. Phosphate component (further shaped body). 4. Calcium component.

FIG. 3 shows dentine surface showing growth.

FIG. 4 shows a SEM image.

It is therefore particularly preferred in this context that the first and/or further shaped body each independently comprise, at least in part, at least one plane arranged on the outer surface or an external envelope. The reduced solubility can be adjusted through covalent chemical cross-linking of a gel and/or through coating of the shaped bodies. The degree of cross-linking and coating can be adjusted freely.

According to a preferred embodiment, the shaped body is arch-shaped and comprises a groove provided to accommodate at least one tooth or at least a part of a dental arch or the shaped body is present in the form of a dental splint or the shaped body corresponds, at least in part, to a negative image of a dental arch or the shaped body adapts in form-fitting or perfectly fitting manner to at least one tooth or a part of a dental arch or corresponds, at least in part, to a negative image of a dental arch or is situated at a distance from a dental arch. The teeth and dental arches corresponds to teeth of vertebrate animals, in particular of humans. Permanent teeth and deciduous teeth are preferred.

According to a particularly preferred embodiment of the invention, the shaped body comprises the following gels: a) the gel of the further shaped body comprises water-soluble phosphates or phosphates that can be hydrolyzed to form water-soluble phosphate ions and comprises a pH value of 2 to 8, and b) the gel of the first shaped body comprises water-soluble calcium ions or compounds releasing calcium ions and comprises a pH value of 3.5 to 14, and a) the gel of the first shaped body and/or b) the gel of the further shaped body comprise (i) a content of water or of a mixture of water and an organic solvent, and (ii), optionally, at least one carboxylic acid and/or a buffer system. According to the invention, b) the gel of the further shaped body contains at least one water-soluble fluoride or a compound releasing fluorides.

The matrix, in particular mineralization matrix or active substance matrix, matrix for release of care products, cosmetics, is based on at least one gel that can be formed during the production and can, in particular, be solidified. Accordingly, the invention comprises a shaped body comprising at least one gel based on an organic gel-forming agent (a) comprising denatured collagen, hydrocolloids, hydrogels, polypeptides, protein hydrolysis products, synthetic polyamino acids, polysaccharides, polyacrylates, polyurethanes, casein, starch flour, cellulose, HPMC, gum arabic, galactomannanes, guar gum, konjac, xanthane, calcium alginate, dextrane, scleroglucan, pectin, carrageenan (κ-, ι- and λ carrageen), Agar-Agar, alginate, alginic acid, sodium alginate, calcium alginate, tragacanth or mixtures comprising at least two of the afore-mentioned gel-forming agents, or inorganic gel-forming agent comprising (b) bentonites, silicic acid gels or mixtures comprising an inorganic gel-forming agent, (c) silicone elastomers, (d) pharmaceutical matrix-forming agents, celluloses, in particular carboxymethylcellulose, acrylates, in particular polyacrylic acids as homo-, copolymers, as block-copolymers or statistical copolymers, as well as mixtures of gel-forming agents (a), (b), (c) and/or (d). It is preferable to use the following gelatine qualities: Bloom 175 to 300 or higher with Gelatine Bloom 300 (pork rind) being preferred. The polysaccharides and/or polyacrylates (eudragit) can preferably also be used, in particular, as second mineralization matrix and/or for formation of the at least one plane or envelope. It is preferable to use gelatine in the matrices to which, according to the invention, a plasticizer such as glycerol or another polyol is added. Adding the plasticizer improves the handling properties of the gelatine.

Shaped bodies according to the invention comprise matrices based on gelatine as gel and preferably a plasticizer, preferably a polyol, such as glycerol and/or conversion products thereof, optionally in the presence of water. Alternatively, gelatine and a plasticizer such as sorbitol can be used just as well. The effect of the plasticizer is to increase the melting range by forming intermolecular hydrogen bonds. According to the invention, gelatine is used as gel, preferably denatured collagen, animal protein, protein; it is particularly preferable to use an acid-hydrolyzed collagen or gelatine and a polyol such as glycerol.

Inorganic gel-forming agents, such as bentonite or silicic acid gels, are also suited. The moulds can be produced, for example by casting the heated matrix after enriching it in the respective active substances, in a two-part tool. Active substances can include: ingredients with a mineralizing effect, such as calcium salts, phosphate salts, and fluoride salts, bleaching agents, antibiotics or other pharmacologically active substances for the dental neck region, plaque-reducing substances, caries-inhibiting substances, etc. The tool allows matrix moulds that correspond to the negative image of the simplified dental arch to be produced. For this purpose, for example, heated gelatine is introduced into the negative mould of the jaw model and the tool is closed, applying light pressure, with the second tool on the opposite side. Cooling-down effects a stabilization of the otherwise very soft matrix in the three-dimensional mould. Further stabilization of the mould is implemented through chemical cross-linking. Further improvements as compared to the system for depositing apatite on hard dental substance described specifically in a parallel patent application can be attained with the present application:

Further simplification of the application is attained in that the calcium-containing gel facing away from the tooth is already introduced in an application dental splint, which is synonymous to protective splint. Said arrangement can be implemented by first introducing the dental splint into the tool and then introducing the mouldable matrix comprising the liquid gel (insertion method). On a large technical scale, this can be implemented, e.g., by means of 2-K injection moulding methods. In this method, both the dental splint and/or protective splint and the shaped body, i.e. gel splint, can be produced directly, one after the other, in one mould. Another option is to insert the mouldable matrix as a warmed-up gel into ready-made application dental splints by extrusion. Moreover, cast gel plates can be re-shaped as required by slight heating. Thermo-deformable and/or thermally deformable dental splints/protective splints, as described in WO 2009045857 A1 and WO 2009029886 A1, are particularly suited. Full reference is thus made to the disclosure of WO 2009045857 A1 and WO 2009029886 A1, in particular to the thermally deformable polymers and the dental splint geometry described therein, which is thus made a content of the present patent application. For the patients, this strongly simplifies the application. Now, only steps 1-3 are required:
1. rinsing the mouth with the pre-treatment
2. inserting the phosphate gel splint
3. inserting the calcium gel-impregnated protective splint, synonymous to application dental splint, and matrix as a unit.

Using the invention, it is feasible to apply soluble active substances to the tooth without these being rinsed off right away. A customisable splint with no further sealing provides sufficient protection. In particular, a complex system for depositing apatite on natural hard dental substance can be made significantly more convenient. The mineralization can act virtually everywhere on the teeth and does not only reach sub-areas of the teeth. Since the application is simple, the system can be used both by dentists on patients and by the patients on themselves. As a matter of principle, the application system can be implemented through the insertion technique or 2K-injection moulding.

The deposition of apatite preferably proceeds by making the matrix, which is composed on the basis of denatured collagen or other gel-forming agents and mineral substances, in particular the mineralization matrix, insoluble in the oral environment by means of chemical modification, in particular as a formulation with separated shaped bodies or as a multi-part shaped body each comprising at least one or two mineralization matrices. Surprisingly, the mineralization activity is affected beneficially despite the modification, since the gel no longer spreads in the oral environment and can act during the entire duration of treatment. Said chemical modification preferably proceeds by means of at least partial chemical cross-linking at the surface of the mineralization matrix such that at least one partially chemically cross-linked plane or partial envelope is formed. Preferably, the at least one plane is a surface of the mineralization matrix; preferably the planes form an outer envelope at the surfaces of the mineralization matrices. According to the invention, the cross-linked planes or the envelope act like a membrane through which aqueous media, such as saliva, can penetrate into the mineralization matrix and, concurrently, apatite can be deposited on the tooth surfaces outside of the mineralization matrix, which contact the at least one plane or the envelope of the mineralization matrix. According to the invention, the plane or envelope formed can, on the one hand, adapt optimally to the surfaces of the teeth when the material swells in the mouth due to it being a very thin layer and, on the other hand, also favours the deposition of apatite at the approximal spaces. Moreover, the mineralization matrix becomes more flexible at body temperature and can optimally adapt to the tooth contours. It is another particular advantage of the superficial cross-linking of the mineralization matrix, during which a shaped body is formed, that an at least partially elastic shaped body is formed which can adapt optimally to the surface contour of teeth and rows of teeth. Due to swelling of the mineralization matrix in the oral environment, the partially elastic shaped body adapts particularly well to the tooth surfaces. In order to affix the shaped body, comprising at least one mineralization matrix, optimally to the buccal, labial, mesial and/or approximal surfaces of the teeth, it is advantageous to place the at least one shaped body in an application dental splint and/or to provide it in the splint. Since the shaped body according to the invention is affixed with a dental splint, apatite can be deposited, at least on part or almost completely, on the teeth of the upper and lower jaw in buccal, mesial, labial, palatinal, distal and approximal position. The use of the at least one shaped body according to the invention allows, easily and for the first time, for biomimetic remineralization of at least, partially, more than or equal to 1 μm, preferably of more than or equal to 2 μm, preferably of more than or equal to 3 μm through inserting the shaped bodies into an application dental splint and placing the splint on the teeth over night, for example for approx. 8 to 12 hours, optionally up to 16 or 24 hours, alternatively during the day as well, whereby biomimetic remineralization of at least partially, preferably on average, from 1 to 10 μm, 1 to 5 μm is particularly preferred. It is particularly preferred for the remineralization to occur in two-dimensional manner in an area and with the thickness of the layer formed being as homogeneous as possible.

Surprisingly, it has been found that the mineralization product forms more evenly and more densely on the tooth surface after the chemical stabilisation. The cross-linked plane or envelope is so porous that comparable apatite layers can be deposited despite the formation of the envelope, as is shown by examples according to the invention. Presumably, the chemical modification leads to the formation of a less temperature-sensitive and preferably less hydrolysis-sensitive network of polypeptides around the mineralization matrix, whose pores are large enough, though, to still allow molecules and ions forming the stable, ordered apatite layer on the tooth surface to pass. The chemically cross-linked layer or plane basically acts like an ion- and molecule-permeable membrane.

A sufficiently thick, homogeneous, and stable apatite layer can be attained by the optimal interplay of the components: mineral salts, buffer, and pH value. It is crucial to select the concentrations correctly at a corresponding degree of cross-linking. The formulation according to the invention can be used for depositing apatite on teeth or any other biological surfaces, such as a bone matrix.

Since the application of the formulations according to the invention is simpler, the system can be used both by dentists on patients and by the patients on themselves. The deposition of a fluoride-rich calcium phosphate layer can reduce sensitivities on the teeth, it can reduce cracks and initial porosities and increases the acid stability of the teeth. Initial losses of hard dental substance due to acid erosion can be stopped and/or partially or completely reversed. The increased fluoride content in the layers as compared to untreated teeth reduces the solubility of the newly formed mineral. The natural enamel is protected from toothbrush erosion by the protective layer.

An object of the invention are shaped bodies that are well-suited for biomimetic deposition of apatite, selected from fluoroapatit (Ca₅[F|(PO₄)₃), hydroxylapatite (Ca₅[F|(PO₄)₃) or mixtures thereof on teeth or on a bone matrix of vertebrates, whereby the shaped body comprises at least one partially three-dimensional and arch-shaped shaped body, comprising at least one matrix, in particular one mineralization matrix, containing at least one gel, and whereby the shaped body preferably comprises reduced solubility with respect to aqueous media, at least in part, in at least one plane as compared to the matrix, in particular mineralization matrix, whereby the plane serves as membrane, and

a) the at least one mineralization matrix comprises a gel containing water-soluble phosphates or phosphates that can be hydrolyzed to form water-soluble phosphate ions and has a pH value of 2 to 8 (phosphate matrix), and
b) the at least one or a second mineralization matrix comprises a second gel having a pH value of 3.5 to 14 comprising calcium ions, or compounds releasing calcium ions (calcium matrix), particularly preferably the at least one mineralization matrix is present in a first shaped body I. and the second mineralization matrix is present in a second shaped body II., alternatively two mineralization matrices are present in one shaped body.

According to a preferred embodiment of the invention, the at least one mineralization matrix or the second mineralization matrix, in particular mineralization matrices, each independently comprising at least one gel are present in the form of an arch-shaped shaped body, in particular first and/or further shaped body, preferably as an at least partial negative image of the jaw. It is preferred that the aforementioned at least one plane is a plane arranged on the outer surface or is an essentially complete outer envelope.

Formulations according to the invention comprise a first or further shaped body in the form of arch-shaped, partially elastic shaped body each having an enclosing envelope having reduced solubility that functions, in particular, as a membrane. The afore-mentioned shaped bodies are preferably present with side walls, which each independently have defined layer thicknesses, preferably in the form of U-, J-, L-shaped profiles. The side walls usually adapt to the teeth in labial, buccal, and/or distal position. The thickness of the layers is advantageously in the range of 5 to 10,000 μm, preferably 10 to 6,000 μm, particularly preferable are mineralization matrices with layer thicknesses of 5 to 3,000 μm, preferably 100 to 600 μm, in particular 300 to 600 μm or about 500 μm plus/minus 100 μm, in particular plus/minus 50 μm. The shaped bodies according to the invention comprise, at least partially, an envelope or at least one plane of reduced solubility. Preferably, the shaped bodies are present in the form of at least one partial negative image of the dental arch or jaw, preferably as a negative mould of the at least one tooth or of teeth of the upper and/or lower jaw. Shaped bodies of the calcium matrix are preferably present having a layer thickness from 500 to 3,000 μm, in particular 500 to 1,500 μm, particularly preferably from 500 to 1,200 μm or about 1,000 μm plus/minus 200 μm, in particular plus/minus 100 μm. Shaped bodies of the phosphate matrix are preferably present at a layer thickness of 100 to 3,000 μm, in particular 100 to 1,000 μm, particularly preferably 150 to 800 μm, preferably 300 to 800 μm, in particular 400 to 600 μm or about 500 μm plus/minus 200 μm, in particular plus/minus 100 μm, in particular plus/minus 50 μm.

Shaped bodies according to the invention have a water content after cross-linking, and optionally after subsequent drying, of 8 to 60% by weight, preferably of 30 to 55% by weight. It is also preferred for the gels of the shaped bodies according to the invention to have a water content after cross-linking, and optionally after subsequent drying, in the mineralization matrix of the phosphate matrix of 20 to 40% by weight, preferably of 25 to 35% by weight, particularly preferably about 30% by weight with a deviation of plus/minus 5% by weight. Accordingly, shaped bodies of the calcium matrix that have a water content after cross-linking, and optionally after subsequent drying, in the mineralization matrix of 30 to 60% by weight, preferably of 40 to 60% by weight, more preferably about 50% by weight with a deviation of plus/minus 5% by weight are preferred. The shaped bodies thus produced can subsequently be sealed in blisters, sachets or the like such as to be air- and moisture-tight.

The envelope or the plane prevents the respective shaped body comprising the matrix and/or mineralization matrix from dissolving in the oral environment. However, H₂O from saliva can penetrate and fluorides, calcium ions, and phosphate ions as well as composites or polypeptides can diffuse through the envelope and can be deposited on the tooth surface as apatite, hydroxylapatite or fluorapatite. The envelope encloses a water-rich gelatine matrix from which the composites, ion-loaded water molecules and/or hydrated ions and/or active substances can diffuse. The planes or the envelope act(s) as membrane for chemical compounds through which the active substances, pharmacologically active substances, care products, composites, for example made of polypeptides and salts, as well as for salts and water, etc., can diffuse. The diffusion of the composites is important since the composites are organic macromolecule-substituted hydroxyapatites that form the enamel, and in order to deposit these on the tooth surfaces. According to the invention, fluorapatite-protein composites from the shaped bodies are deposited on the tooth surfaces. Reduced solubility of the chemically cross-linked plane or envelope with respect to aqueous media as compared to the mineralization matrix shall be understood as follows: the mineralization matrix not chemically cross-linked and, optionally, the mineralization matrix modified with a plasticizer only, for example the gelatine cross-linked to glycerol, preferably as adduct, via hydrogen bonds.

According to the invention, the at least one plane or the envelope form the outer boundary of at least one matrix such as the mineralization matrix. The plane or envelope can be obtained in a variety of ways by chemically cross-linking the mineralization matrix or by applying a coating to the mineralization matrix in order to form a mineralization matrix surface that is permeable for ions and water and acts as membrane. The cross-linking or coating can be effected by immersing, application techniques, such as painting, spraying, rolling, and other measures known to a person skilled in the art. The planes or the envelope can just as well be formed to have irregular or regular perforations and/or pores or pore-forming agents can be added.

The fluorapatite deposited from the shaped bodies according to the invention is preferably present on the teeth in crystalline form, preferably micro-crystalline, particularly preferably in the form of needle-shaped crystallites. The at least partial apatite layer, preferably contiguous apatite layer, deposited on the tooth surfaces in the course of one application cycle of approx. 8 hours has a layer thickness of at least 2 μm. The scope of the invention also includes at least partially non-contiguous apatite layers that cover at least a part of the treated tooth surface irregularly to preferably homogeneously. The apatite layers can, at least in part, be up to 15 μm, preferably essentially homogeneous, which is preferred, apatite layers of more than or equal to 2 μm, more preferably more than or equal to 5 μm to 13 μm, and on average of 2 to 10 μm are obtained.

Teeth of vertebrates include human teeth, prostheses of human teeth, deciduous teeth (Dentes decidui), permanent teeth (Dentes permanentes), crowns, inlays, implants, teeth of animals, such as domestic and livestock animals, such as dogs, horses, cats.

In the scope of the invention, an at least partially elastic shaped body shall be understood to mean a three-dimensional shaped body that has elastic properties. According to the invention, the shaped body is arch-shaped, in particular in the form of at least a partial negative image of the jaw, and has elastic properties. The shaped body shall be considered to be elastic or partially elastic if the body changes its shape when exposed to a force and returns to its original shape, partly or fully, when the force ceases to act on it. The shaped body preferably possesses the property of being elastic or partially elastic when it is applied in the oral environment and preferably after production. The elasticity may decrease upon excessive drying.

In a further shaped body that is particularly preferred according to the invention, the mineralization matrix comprises in a) the following composition: a) the at least one mineralization matrix comprises a gel comprising (i) water-soluble phosphates or phosphates that can be hydrolyzed to form water-soluble phosphate ions, in particular Na₂HPO₄, whereby the phosphate content in the mineralization matrix preferably is 1 to 10% by weight, more preferably 2 to 8% by weight, particularly preferably 5 to 8% by weight, (ii) a content of water or of a mixture of water and an organic solvent, (iii) optionally at least one carboxylic acid, in particular a hydroxycarboxylic acid, such as lactic acid, and/or a buffer system, in particular a buffer system is present for adjusting the pH value in the range of 2 to 8, in particular from 3.5 to 8, preferably from 3.5 to 6, particularly preferably 5.5 plus/minus 0.5. The content refers to PO₄³⁻.

Concurrently, the particularly preferred first shaped body according to the invention comprises in b) the following composition: the second mineralization matrix or the at least one mineralization matrix comprises a second gel comprising (i) calcium ions or compounds releasing calcium ions, in particular calcium dichloride or hydrates thereof, preferably in addition calcium sulfate, nanoapatite, sodium carbonate or calcium oxalate, whereby the calcium content in the mineralization matrix preferably is 1 to 10% by weight, more preferably more than or equal to 1.5 to 7.5% by weight, (ii) optionally water or a mixture of water and an organic solvent, and (iii) optionally at least one carboxylic acid, such as a hydroxycarboxylic acid, for example lactic acid, and/or a buffer system. It is preferred to use fruit acids and alkali salts to produce the buffers. The content refers to calcium (Ca²⁺).

Moreover, it is preferred that the formulation comprises a gel in the at least one matrix, whereby the gel comprises at least one water-soluble fluoride (F⁻), with fluoride ions, or a compound releasing fluorides. Particularly preferably, the further shaped body in a) comprises as further component (iv) at least one water-soluble fluoride or a compound releasing fluorides. According to an alternative, the matrix of the first shaped body can comprise fluorides as active substance for fluoridation of the teeth, meaning that the shaped body serves as a system for release of an active substance and in particular does not serve for deposition of apatite.

According to a preferred refinement of the invention, the at least one water-soluble fluoride or the at least one compound releasing fluorides comprises, in particular in a), the at least one mineralization matrix, (i) at least one non-substituted or substituted alkyl groups-comprising quaternary mono- or poly-ammonium compound, preferably having four substituted alkyl groups, whereby the at least one substituted alkyl group comprises hydroxyalkyl, carboxyalkyl, aminoalkyl groups having 1 to 25 C-atoms or organo-functional, hetero atom-interrupted groups having up to 50 C-atoms. Preferred ammonium compounds can contain 1 to 20 quaternary ammonium functions, preferably 1, 2, 3, 4, 5, 6, 7, 8 ammonium functions; it is preferable to use Olaflur (N,N,N′-tris(2-hydroxyethyl)-N′-octadecyl-1,3-diaminopropandihydrofluoride) as water-soluble fluoride. Also preferred are aminefluorides, such as Oleaflur, Decaflur (9-Octadecenylaminhydrofluoride), ethanolamine hydrofluoride, (ii) a fluorides-releasing organo-functional amino compound or a fluorides-releasing antiseptic based on organo-functional amino compounds, such as, in particular, fluorides of N-octyl-1-[10-(4-octyliminopyridin-1-yl)decyl]pyridin-4-imine, cetylpyridinium fluoride, or c) water-soluble inorganic fluorides, such as alkali fluorides, sodium fluoride, potassium fluoride, tin fluoride, ammonium fluoride, or fluorides-releasing inorganic fluorides, such as zinc fluoride, zinc hydroxyfluoride.

The chemical cross-linking, in particular covalent cross-linking, for formation of the at least partially chemically cross-linked plane or partially cross-linked envelope comprising reduced solubility with respect to aqueous media as compared to a matrix that is not chemically cross-linked in this way preferably is effected by cross-linking with cross-linkers such as dialdehydes, whereby it is preferred that all external surfaces of the mineralization matrix are chemically cross-linked at least partially. The formation of just intermolecular hydrogen bonds due to the presence of glycerol in the gelatine improved the handling properties and thermal stability of the gelatine. Shaped bodies according to the invention can just as well comprise a surface texture that simulates the tooth surfaces of a dental arch.

The di- or poly-functional cross-linkers, preferably glutardialdehyde, are used as compounds for chemical cross-linking, in particular covalent cross-clinking, in the at least one plane or for forming the envelope of the matrix. The chemical cross-linkers form covalent cross-linking sites with the gels, in particular with the polypeptides or polyamino acids. Preferably, the di- or poly-functional cross-linkers comprise dialdehydes, polyepoxides and/or polyisocyanates as well as mixtures comprising at least two cross-linkers. Furthermore, it is preferred to use pharmacologically tolerable cross-linkers. Preferred dialdehydes comprise alpha, omega dialdehydes of hydrocarbons, in particular comprising 2 to 50 C-atoms, in particular 4 to 10 C-atoms in the di-functional alkylene group. Treating the mineralization matrix with a cross-linker reduces the solubility of the gelatine to a level such that it does not liquefy in the oral environment for approximately 8 hours. Preferably, the treatment with glutardialdehyde proceeds for at least 5 s, depending on the application on hand the cross-linking may proceed for longer and thus be more pronounced, for example if a mineralization matrix is to remain in the oral environment for 12 to 16 hours. After rinsing for 40 s in an 0.5% glutardialdehyde solution, the solubility of the gel is reduced to a level such that it does not liquefy in the oral environment for up to 8 hours. The membrane (layers) that can be used optionally are free of ions.

The cross-linker solution preferably has a cross-linker content of approx. 0.25 to 0.5% by weight, preferably of glutardialdehyde. It has been evident that the best results in terms of sufficient cross-linking of the shaped bodies and optimal permeability for the apatite composites to be deposited are obtained with a treatment time of 0 to 60 s, preferably approx. 5 to 40 s, particularly preferably 10 to 30 s, according to the invention about 20 seconds (s).

According to a preferred embodiment of the invention, one subject matter of the invention is an application system for treating teeth of vertebrates, in particular for dental treatment of one or more human teeth, comprising at least one application dental splint, having at least one shaped body according to the disclosure of the invention, whereby the application splint is formed and/or suitable to accommodate at least one first three-dimensional shaped body that is adapted to the splint and contains a matrix. According to the invention, the application dental splint can be deformed by thermal means. According to a preferred embodiment of the application system, at least one further three-dimensional shaped body can be inserted into the first shaped body, whereby the further shaped body contains a second matrix. The application system advantageously comprises a first shaped body and a further shaped body, whereby the further shaped body is provided to accommodate at least one tooth or a part of a dental arch when the application dental splint is used by a vertebrate.

The application dental splint according to the invention is preferably present in the form of a U-shaped, J-shaped or L-shaped profile, in particular the splint is arch-shaped along the profile, or the application dental splint is selected from an arch-shaped dental splint with a groove, which preferably is provided to accommodate at least one tooth or at least a part of a dental arch, whereby the dental splint corresponds, at least in part, to a negative image of a dental arch, the dental splint is situated, at least in part, at a distance to the partial dental arch, the dental splint is situated, at least in part, at a distance to the partial dental arch and comprises a reservoir for accommodation of the first and/or further shaped body, the dental splint corresponds to at least one tooth or the dental splint corresponds, at least in part, to a negative image of a jaw.

According to a preferred embodiment of the invention, the application dental splint and the first shaped body are a unit in the application system, preferably the first shaped body and the dental splint are firmly connected to each other. The matrix of the first shaped body advantageously comprises active substances or forms the calcium matrix in said embodiment. The calcium matrix can be assembled with the further shaped body comprising the phosphate matrix at a later time to form an application system for biomimetic deposition of apatite, in particular fluorapatite.

According to the invention, the matrix of the application dental splint, the matrix of the first shaped body or the matrix of the further shaped body comprises at least one active substance or a system having a mineralizing effect, including phosphates, calcium, and fluorides, pharmacologically active substances comprising antibiotics, antiseptic agents, active substance promoting the oral mucosa, bleaching agents, plaque-reducing substance, caries-inhibiting and/or caries-reducing substance.

Both the shaped body alone and the shaped body that can be inserted in the application system is selected from a first and further shaped body, whereby each shaped body independently is arch-shaped and comprises a groove that is provided to accommodate at least one tooth or at least a part of a dental arch, the shaped body is present in the form of a dental splint, the shaped body corresponds at least in part to a negative image of a dental arch, the shaped body adapts in form-fitting or perfectly fitting manner or is situated, at least in part, at a distance from said partial dental arch or at least one tooth, the shaped body corresponds, at least in part, to a negative image of a jaw.

According to an alternative embodiment of the invention, the application system comprises, alternatively, (i) a) an application dental splint that accommodates a first three-dimensional shaped body containing a matrix, and b) the first three-dimensional shaped body accommodates a further shaped body containing a matrix, optionally separated by a membrane layer, or, according to an alternative, (ii) a) the application dental splint and the first shaped body are a unit, whereby b) the first three-dimensional shaped body accommodates a further shaped body containing a matrix, optionally separated by a membrane layer, or (iii) a) the application dental splint comprises a matrix and accommodates a first three-dimensional shaped body containing a matrix, optionally separated by a membrane layer.

The application dental splint of the application system is formed, at least in part, from a polymer comprising thermoplastic polymers, elastomers, thermoplast, thermoplastic elastomers, duroplastic polymers and/or from a mixture containing at least two of the afore-mentioned polymers and, optionally, further common filling agents, rheology-modifying agents, additives, cross-linkers and/or catalysts. Also preferred are (i) thermoplastic polymers comprising homo- and/or copolymers comprising acrylonitrile-butadiene-styrene (ABS), acrylonitrile-ethylene-butadiene-styrene, polyamides (PA), polylactate (PLA), polymethylmethacrylate (PMMA), polycarbonate (PC), polyethyleneterephthalate (PET), polyethylene (PE), high density polyethylene (HDPE), low density polyethylene (LDPE), ultra-low density polyethylene (ULDPE), polypropylene (PP), polystyrene (PS), polyetheretherketone (PEEK), ethylenevinylacetate (EVA), (ii) the elastomer-forming polymers comprise silicones, alginate, rubbers, natural rubber, and silicone rubber, or (iii) thermoplastic elastomers comprise olefin-based thermoplastic elastomers, PP/EPDM, olefin-based cross-linked thermoplastic elastomers, such as PP/EPDM, urethanes, thermoplastic polyesterelastomers, co-polyesters, styrene-block copolymers such as SBS, SEBS, SEPS, SEEPS, and MBS, thermoplastic co-polyamides and mixtures comprising at least two of the afore-mentioned compounds.

Examples of elastomers comprise natural rubbers, polyisoprenes, butyl rubbers, isobutylene, and isoprene-copolymers, halogenated butyl rubbers, chlorobutyl rubber, bromobutyl rubber, polybutadienes, polystyrene, and polybutadiene-copolymers, nitrile rubbers, hydrogenated nitrile rubbers, polybutadiene and acrylonitrile-copolymers, chloroprene rubbers, polychloroprene, neoprene, polyethylene-saturated rubbers, ethylene-propylene rubbers and polypropylene-copolymers, ethylene-propylene-diene rubbers, epichlorohydrin rubbers, polyacrylo rubbers, silicone rubbers, fluorosilicone rubber, perfluoro elastomers, polyether-block amides, tetrafluoroethylene rubbers/propylene rubbers, chlorosulfonated polyethylenes, ethylene vinylacetates, thermoplastic elastomers, thermoplastic vulcanisation products, polyurethane rubbers, polysulfide rubbers, silicones, polyorganosiloxanes, combinations thereof and the like. In one embodiment, the elastomer can be a silicone. Silicone materials are generally compressible and thus are softer. At body temperature, silicones are resistant, tenacious, flexible, and tough. On the other hand, silicones are advantageous in that they can make shaped bodies that are thin, but stable in shape or, optionally, application dental splints or parts thereof, such as internal linings. Moreover, silicones are relatively stable and take up little moisture. Silicones comprise homo- and copolymers that contain at least one siloxane of dimethylsiloxanes, methylphenylsiloxanes, methylvinylsiloxanes, methylfluoroalkylsiloxanes, methylethylidennorbornene siloxanes enthalten. Preferred polyorganosiloxanes comprise silicones, polydimethylsiloxanes, polymethylphenylsiloxanes, polydialkylsiloxanes, polymethylvinylsiloxanes, polymethylfluoroalkylsiloxanes, polymethylethylidennorborn-nesiloxanes or mixtures containing them. In addition, the membrane can be made from materials that are customary in dental applications, such as polyolefins, ethylene-vinylacetate copolymer (EVA), ethylene-vinylalcohol copolymer (EVAL), polycaprolactone (PCL), polyvinylchloride (PVC), polyester, polycarbonate, polyamide, polyurethane, polyesteramide, cellulose ethers, ethyl cellulose, cellulosepropyl, isopropyl cellulose, cellulose butyl, t-butyl cellulose, cellulose acetate, polyvinylacetate, polyvinylalcohol, shellac, chemically or light-curing materials (e.g. methacrylate or acrylate resins), combinations thereof and the like. Suitable polyolefins for production of the application dental splint comprise polyethylene (PE), high density polyethylene (HDPE), low density polyethylene (LDPE), ultra-low density polyethylene (ULDPE), polypropylene (PP), and polytetrafluoroethylene (PTFE) (e.g. Teflon). Vinyl-terminated siloxane polymer mixtures are typically heat-cured. They contain benzoylperoxide that acts as an initiator of radical polymerisation and cross-linking. In one embodiment, the shaped body can comprise hydroxyl-terminated polydimethylsiloxane cured by condensation reaction. As catalyst or cross-linker, tetraethylsilicate and a catalyst, such as, e.g., dibutyl tin dilaurate, can be added. The cross-linked polysiloxane is a highly elastic elastomer.

An application system according to the invention comprises (i) the further shaped body containing a matrix, whereby the further three-dimensional shaped body is at least partially elastic and, in particular, is arch-shaped, and the (a) matrix (phosphate matrix) comprises (a1) at least one mineralization matrix comprising at least one gel, comprising (a2) at least one water-soluble phosphate or phosphates that can be hydrolyzed to form water-soluble phosphate ions, and (a3), optionally, at least one carboxylic acid and/or a buffer system, (a4), optionally, water-soluble fluorides or a compound releasing fluorides, (a5), optionally, a content of water or of a mixture of water and an organic solvent, and/or (ii) the first shaped body containing a matrix, and the first three-dimensional shaped body is at least partially elastic and, in particular, is arch-shaped, and the (b) matrix (calcium matrix) comprises (b1) at least one mineralization matrix comprising at least one gel, comprising (b2) water-soluble calcium ions or compounds releasing calcium ions, and (b3), optionally, at least one carboxylic acid and/or a buffer system, (b4), optionally, water or a mixture of water and an organic solvent, or the first shaped body comprises matrices and is at least partially elastic, whereby the (c) matrices comprise (c) partially elastic shaped body C comprises (c1) at least one mineralization matrix comprising at least one gel, containing (c1.1) at least one water-soluble phosphate or phosphates that can be hydrolyzed to form water-soluble phosphate ions, and (c1.2), optionally, at least one carboxylic acid and/or a buffer system, (c1.3), optionally, water-soluble fluorides or a compound releasing fluorides, (c1.4), optionally, a content of water or of a mixture of water and an organic solvent, (c2), optionally, a membrane (layer), and (c3) at least one mineralization matrix comprising at least one gel, containing (c3.1) water-soluble calcium ions or compounds releasing calcium ions, and (c3.2), optionally, at least one carboxylic acid and/or a buffer system, (c3.3), optionally, water or a mixture of water and an organic solvent, whereby the layer structure of the shaped body is c1 and c3 or c1, c2, and c3, whereby c3 adapts to the application dental splint and c1 is arranged on the inside, in particular in order to contact at least one tooth or a part of a dental arch during a dental application in a vertebrate.

The invention also comprises an application system, whereby in an alternative (i) the application dental splint comprises a first three-dimensional shaped body containing a matrix and the first shaped body and the application dental splint are a unit, whereby (b) the matrix comprises (b1) at least one mineralization matrix comprising at least one gel, (b2) water-soluble calcium ions or compounds releasing calcium ions, and (b3), optionally, at least one carboxylic acid and/or a buffer system, (b4), optionally, water or a mixture of water and an organic solvent, or (ii) the first shaped body (b) comprising the mineralization matrix (b1) with (b2), (b3) and, optionally, (b4) can be inserted into the application dental splint.

Another subject matter of the invention is an application system, whereby, in an alternative (i), the application dental splint comprises a first shaped body into which a further shaped body containing a matrix can be inserted, whereby the further shaped body is three-dimensional, and the (a) matrix comprises (a1) at least one mineralization matrix comprising at least one gel, comprising (a2) at least one water-soluble phosphate or phosphates that can be hydrolyzed to form water-soluble phosphate ions, and (a3), optionally, at least one carboxylic acid and/or a buffer system, (a4), optionally, water-soluble fluorides or a compound releasing fluorides, (a5), optionally, a content of water or of a mixture of water and an organic solvent, or (ii) the application dental splint and the further shaped body (a) comprising the mineralization matrix (a1) with (a2), (a3), optionally (4a), and, optionally, (a5), optionally separated by a membrane, can be present as a unit as disclosed above.

The carboxylic acids are preferably selected from fruit acids, such as α-hydroxycarboxylic acids such as malic acid, citric acid, glycolic acid, lactic acid, and tartaric acid; amino acids, fatty acids, hydroxycarboxylic acids, dicarboxylic acids, and mixtures comprising at least two of the aforementioned acids and/or the buffer system comprises carboxylates of alkylcarboxylic acids, fatty acids, fruit acids, fumarates, amino acids, hydroxycarboxylic acids, dicarboxylic acids, and mixtures comprising at least two of the aforementioned acids or phosphate buffer. It is advantageous to use alkali and/or alkaline earth salts or zinc salts for the buffer systems.

The buffer systems comprise EDTA, TRIS: tris(hydroxymethyl)-aminomethane for pH 7.2 to 9.0, HEPES: 4-(2-hydroxyethyl)-1-piperazinethanesulfonic acid for pH 6.8 to 8.2, HEPPS: 4-(2-hydroxyethyl)-piperazin-1-propansulfonic acid for pH 7.3 to 8.7, barbital-acetate buffer, MES: 2-(N-morpholino)ethansulfonic acid for pH 5.2 to 6.7, carbonic acid-bicarbonate system for pH 6.2 to 8.6; neutral, carbonic acid-silicate buffer for pH 5.0 to 6.2; weakly acidic, acetic acid-acetate buffer for pH 3.7 to 5.7, phosphate buffer: NaH₂PO₄+Na₂HPO₄ for pH 5.4 to 8.0, ammonia buffer NH₃+H₂O+NH₄CI for pH 8.2 to 10.2, citric acid or citrate buffer. Particularly preferred buffer systems comprising lactic acid buffer systems, EDTA, or barbital-acetate buffer and, in the mouthwash, TRIS (tris(hydroxymethyl)-aminomethane) buffer. TRIS (Tris(hydroxymethyl)-aminomethane) is used in the mouthwash, which is a synonymous term for pre-treatment solution.

Phosphates that can be used according to the invention to produce the phosphate-containing mineralization matrices comprise phosphates, hydrogenphosphates or phosphates that can be hydrolyzed to form water-soluble phosphate ions, comprising a) alkali phosphates, alkaline earth phosphates, dihydrogenphosphates, sodium dihydrogenphosphate, NaH₂PO₄, potassium dihydrogenphosphate, KH₂PO₄, hydrogenphosphates, dipotassium hydrogenphosphate, K₂HPO, disodium hydrogenphosphate, Na₂HPO₄, phosphate esters, monoesters, diesters, and triesters of phosphates, sodium phosphate, Na₃PO₄, potassium phosphate, K₃PO₄, calcium dihydrogenphosphate, Ca(H₂PO₄)₂, monoesters, diesters, and triesters calcium hydrogenphosphate, CaHPO₄, calcium phosphate, Ca₃(PO₄)₂ and/or b) the calcium ions or compounds releasing calcium ions comprise calcium chloride, calcium dichloride dihydrate, calcium salt of a carboxylic acid comprising alkyl carboxylic acids, hydroxy carboxylic acid, dicarboxylic acids, fruit acids, amino acids, such as calcium lactate, calcium gluconate, calcium lacto-gluconate, calcium alginate, calcium L-ascorbate, compounds releasing poorly water-soluble calcium ions in delayed manner comprising calcium sulfate, calcium apatite, calcium carbonate, calcium oxalate, calcium phosphate, calcium alginate, preferably having a particle size of less than 100 μm, preferably about 10 μm, particularly preferably of less than or equal to 5 μm, for example up to 1 μm or 50 nm, or preferably mixtures of water-soluble and poorly water-soluble calcium ions or compounds releasing calcium ions. The poorly water-soluble compounds releasing calcium ions in delayed manner are added to the gel containing calcium ions that are easily soluble in water in order to improve the texture of the, in some cases, tacky gels. A total of 1 to 50% by weight compounds releasing poorly water-soluble soluble calcium ions, preferably 5 to 30% by weight with respect to the total composition of the mineralization matrix, can be used.

According to an embodiment of the invention, a formulation for producing an aqueous pre-treatment solution, which is synonymous to mouthwash, for pre-treating the teeth is disclosed that contains at least one calcium salt that dissolves well in water, preferably comprising calcium lactate, calcium chloride, calcium gluconate, calcium lacto-gluconate, a hydrate of the salts or a mixture containing at least two of the salts, optionally a content of a buffer system, in particular TRIS (tris(hydroxymethyl)-aminomethane), optionally a content of masking agent or flavouring agent and common formulation excipients for producing a pre-treatment solution or together with water as pre-treatment solution prior to a pre-treatment, in which apatite is deposited on vertebrate teeth. TRIS (Tris(hydroxymethyl)-aminomethane) is used in the mouthwash (pre-treatment solution). Also a subject matter of the invention is a formulation in the form of an aqueous mouthwash comprising water, 0.01 to 2 mol of a calcium salt that dissolves well in water, with respect to the total composition, optionally 0.01 to 0.5 mol, preferably 0.05 to 0.2 mol of a buffer system, in particular TRIS (tris(hydroxymethyl)-aminomethane), optionally masking agent or flavouring agent, and having a pH value of 5.0 to 12.0. The mouthwash or pre-treatment solution can also be used to treat single teeth.

According to another alternative embodiment, the invention discloses a formulation of an aqueous mouthwash, preferably for use in combination with any afore-mentioned formulation according to the invention, comprising water, 0.1 to 30% by weight of a calcium salt that dissolves well in water, in particular 5 to 20% by weight, preferably 5 to 15% by weight, with respect to the total composition, preferably comprising calcium lactate, calcium chloride, calcium gluconate, calcium lacto-gluconate, a hydrate of the salts or a mixture containing at least two of the salts, optionally a content of a buffer system, in particular Tris, optionally masking agent or flavouring agent and comprising a pH value of 5.0 to 12.0.

Also a subject matter of the invention is a method for producing the shaped body and a shaped body that can be obtained according to the method, in that a mouldable matrix is introduced into a first work piece, in particular one having a recess, that corresponds to at least a partial negative image of a jaw or partial negative image of a dental arch or is derived from either, and is formed together with a second work piece that corresponds, at least in part, to a positive image of a jaw or partial positive image of a dental arch, at normal pressure or excess pressure, optionally is being cooled, whereby the formed matrix is taken out as a shaped body after forming, whereby the first or the further shaped body is obtained. The production of the mouldable matrices is described in the following. The cross-linking can be performed subsequently.

Also a subject matter of the invention is a method for producing the application system and an application system that can be obtained according to the method, in which (a) an application dental splint (a.1) is provided or (a.2) formed, in particular by means of injection moulding, extrusion or other method known to a person skilled in the art, the dental splint is preferably formed from the afore-mentioned polymers, such as polyolefins, (b.1) the first shaped body is introduced into the application dental splint (b.1.1) in pre-formed manner, in particular is formed by means of injection moulding of the mouldable matrix, and introduced into the application dental splint, or (b.1.2) in particular, the mouldable matrix is introduced into the application dental splint and formed by means of 2K injection moulding, or (b.1.3) the first shaped body is formed by the user and is introduced into the application dental splint.

The invention also discloses a method in which (c.1) the further shaped body is introduced into the application dental splint (c.1.1) in pre-formed manner, preferably the mouldable matrix is formed, preferably by means of injection moulding, and introduced into the first shaped body in the application dental splint, or (c.1.2) in particular, the mouldable matrix is introduced into the first shaped body by means of 2K injection moulding, optionally separated by means of a membrane, or (c.1.3) the further shaped body is formed by the user and is introduced into the first shaped body in the application dental splint.

According to the invention, the first and/or the further shaped body is produced in said method, in which the mouldable matrix is introduced into a first work piece of at least a partial negative image of a jaw or partial negative image of a dental arch and is formed together with a further work piece, which corresponds, at least in part, to a positive image of a jaw or partial positive image of a dental arch, at normal pressure or excess pressure, optionally is being cooled, the formed matrix is taken out as a shaped body after forming, whereby the first work piece optionally contains the application dental splint when the first shaped body is produced, and, whereby the further work piece optionally also contains an application dental splint during the production of the first shaped body.

Also a subject matter of the invention is a method for producing the shaped bodies or the shaped body for use with the application dental splint, in which

a) the further shaped body comprising a matrix comprising at least one gel containing at least one water-soluble phosphate or compound releasing phosphate, gelatine and glycerol, optionally at least one carboxylic acid and/or a buffer system, optionally water-soluble fluorides or a compound releasing fluorides, optionally a content of water or of a mixture of water and an organic solvent, is produced in that
gel, in particular warm, non-solidified gel, is introduced into the first work piece as mouldable matrix, is pressed under pressure, is cooled optionally, the solidified gel is taken out as matrix or as shaped body comprising the matrix, preferably the matrix is subsequently chemically cross-linked on at least one outer plane or chemically cross-linked on the outside into an envelope, or coated and/or
   b) the first shaped body comprising a matrix comprising at least one gel containing water-soluble calcium ions or compounds releasing calcium ions, optionally at least one carboxylic acid and/or a buffer system, optionally water or a mixture of water and an organic solvent, is produced in that gel, in particular warm, non-solidified gel, is introduced into the second work piece as mouldable matrix, is pressed under pressure, is cooled optionally, the solidified gel is taken out as matrix or as shaped body, preferably the matrix is subsequently chemically cross-linked on at least one external plane or chemically cross-linked on the outside into an envelope or is coated in a plane or with an envelope.

The production of the shaped body of the phosphate matrix comprises, a) preparing, for producing at least the matrix containing the gel, also called phosphate component A, in a first step, a mixture of (i) 0.05 to 4 mol/l, 0.5 to 1.5 mol/l water-soluble phosphates or phosphates that can be hydrolyzed to form water-soluble phosphate ions, (ii) a corresponding amount of water or of a mixture of water and an organic solvent, (iii), optionally, at least one carboxylic acid and/or a buffer system, in particular for adjusting a pH value of 2 to 8, preferably 3.5 to 8, more preferably 3.5 to 6, particularly preferably about 5.5 plus/minus 0.5, (iv) 0 to 6,000 ppm by weight water-soluble fluoride or a compound releasing fluorides, in particular 1 to 4,000 ppm by weight, more preferably 500 to 2,500 ppm by weight, particularly preferably about 2,000 ppm by weight plus/minus 500 ppm by weighed, and in a further step, the mixture produced in a) is used b) with gelatine and, optionally, glycerol while heating to produce the gel while obtaining the mouldable matrix, followed c) by the forming of the matrix, in particular mineralization matrix, and optionally, solidification, and in an optional subsequent step d), a plane, in particular the envelope, of the at least one mineralization matrix that is arranged on the outer surface is formed while the shaped body is being formed.

The plane or envelope can be produced through cross-linking or through application of a coating. For cross-linking the at least one plane of the at least one mineralization matrix that is arranged on the outer surface, in particular in the form of a flat element (e.g. two-dimensional element, element of area) or at least partial negative image of a jaw, the plane is contacted in a further step to a mixture, preferably an aqueous solution, containing a di- or polyfunctional cross-linker.

The phosphate solution for producing phosphate component A contains, inter alia, a water-soluble phosphate salt. For example alkali salts, such as sodium or potassium phosphates, hydrogen or dihydrogen phosphates are well-suited. The listing is inclusive, but not exclusive. The concentration of the phosphate salts in the solution is between 0.05 and 4 mol/l Gel, preferably 0.5 to 1.5 mol/l, particularly preferably about 1 mol/l plus/minus 0.5 mol/l. In addition, the phosphate solution contains a water-soluble fluoride salt, e.g. an alkali salt, or tin fluoride or Olafluor. The listing is inclusive, but not exclusive. The concentration of the fluoride in the solution is between 0 and 6,000 ppm by weight, preferably 200 to 4,000 ppm by weight, particularly preferably 2,500 to 4,000 ppm by weight or about 3,000 ppm by weight plus/minus 500 ppm by weight. The phosphate solution can be used as a cross-linker solution, e.g. upon adding the cross-linker, for example glutardialdehyde.

The pH value of the phosphate solution is between 2.0 and 8.0, preferably between 3.5 and 5,5, and is adjusted using a suitable buffer system. Carboxylic acids, such as ascorbic acid, pyruvic acid, tartaric acid, acetic acid, lactic acid or malic acid, or their water soluble salts can be used as buffer system, but all other buffer systems just as well, are particularly well-suited. The concentration of the buffer is between 0.25 and 4.0 mol/l, preferably between 0.5 and 1.5 mol/l.

The solution is used to produce a gelatine-glycerol gel. The amount of gelatine preferably is 25 to 40% by weight and the amount of glycerol is 5 to 20% by weight with respect to the total composition of aqueous gel. In order to mix the components homogeneously, the preparation is heated to 40 to 90° C., preferably to 50 to 70° C. The thickness of phosphate component A in this context is 50 to 3,000 μm, preferably 200 to 2,000 μm, particularly preferably 300 to 1,500 μm.

The production of the shaped body of the calcium matrix comprises, a) for producing at least the shaped body comprising a matrix containing the gel, also called calcium component B, in a first step, preparing a mixture of (i) 0.1 to 2 mol/l calcium ions or compounds releasing calcium ions, (ii) a corresponding amount of water or of a mixture of water and an organic solvent, (iii), optionally, at least one carboxylic acid and/or a buffer system, in particular for adjusting the pH value to 3.5 to 14, preferably 4.0 to 6.0 or 6.0 to 11.0, preferably 4.0, particularly preferably about 4.0 plus/minus 0.5, and, in a further step, the mixture produced in a) is used b) with gelatine and, optionally, glycerol while heating to produce the gel while obtaining the mouldable matrix, followed c) by the forming of the mouldable matrix, optionally solidification, while obtaining the shaped body, and in an optional subsequent step d), a plane, in particular the envelope, of the at least one mineralization matrix that is arranged on the outer surface is formed while the shaped body is being formed. The plane or envelope can be produced through the aforementioned cross-linking or through application of a coating. A certain degree of porosity is crucial in the production of the plane or envelope in order to enable the deposition of the bio-composites.

For producing the aforementioned formulations, in b), 5 to 50% by weight gelatine with respect to the total composition of the gel and 0 to 30% by weight glycerol with respect to the total composition of the gel are added each independently in a further step, preferably 25 to 40% by weight gelatine and 5 to 20% by weight glycerol are added to produce the formulation containing the matrix containing water-soluble phosphates or phosphates that can be hydrolyzed to form water-soluble phosphate ions, and 20 to 40% by weight gelatine and 15 to 25% by weight glycerol are added to produce the formulation containing the mineralization matrix containing calcium ions or compounds releasing calcium ions.

The respective gelatines in step b) are preferably heated to 40 to 90° C. in order to homogeneously mix the components, whereby the temperature range of 50 to 70° C. is preferred for producing the mouldable matrices.

The solution for producing the calcium component or the shaped body contains a water-soluble calcium salt, e.g. calcium chloride or calcium lactate or calcium gluconate or calcium lacto-gluconate. The listing is inclusive, but not exclusive. The concentration is between 0.1 and 2.0 mol/l, preferably between 0.5 and 1.5 mol/l. The pH value between 4.0 and 14.0, preferably between 6.0 and 11.0, is adjusted using a suitable buffer system. Carboxylic acids, such as ascorbic acid, pyruvic acid, tartaric acid, acetic acid, lactic acid or malic acid, or their water soluble salts can be used as buffer system, but all other buffer systems with a suitable pKs just as well, are particularly well-suited. The concentration of the buffer is between 0.1 and 3.0 mol/l, preferably between 0.25 and 1.0 mol/l. The solution is used to produce a gelatine-glycerol gel. The amount of gelatine preferably is 20 to 40% by weight with respect to the total composition of aqueous gel and the amount of glycerol is 15 to 25% by weight. Since the calcium-gelatine solution is very tacky even after gelling and thus is unpleasant to handle, a poorly soluble calcium salt is added to improve the texture. Calcium sulfate, calcium apatite, calcium carbonate, calcium oxalate are particularly well-suited. The listing is inclusive, but not exclusive. In order to obtain a particularly homogeneous paste, it is advantageous for the particle sizes to be less than 10 μm. It is preferred to use particles with particle sizes of less than 1 μm. The amount of the poorly soluble calcium salt added preferably is 1 to 50%, very preferably 5 to 30%. In order to mix the components homogeneously, the preparation is heated to 40-90° C., preferably to 50 to 70° C., while stirring. The thickness of the first shaped body or of the calcium matrix in this context is 10 to 3,000 μm, preferably 100 to 1,500 μm, particularly preferably 300 to 1,500 μm, even more preferably 500 to 1,500 μm. In order to produce the shaped bodies, the not-yet-solidified gels are formed as mouldable matrices and then solidified. Therefore, also a subject matter of the invention is a method, in which the gel produced in further step d) is formed as mouldable matrix that can be solidified. The shaped bodies can be taken out after cross-linking, and optionally after subsequent drying to a water content between 8 and 60% by weight, preferably 30 to 55% by weight. The drying further stabilises the shaped bodies. The degree of cross-linking can be adjusted by means of the type and concentration of cross-linker, the pH value of the cross-linker solution, and the time of action.

Also a subject matter of the invention is an application system comprising an application dental splint that can be deformed thermally prior to or during the application by a vertebrate animal.

Also a subject matter of the invention is a kit comprising an application system comprising an application dental splint, a first shaped body and/or a further shaped body as well as, optionally, a pre-rinsing solution.

Also a subject matter of the invention is the use of the application system according to the invention or of the kit according to the invention for biomimetic deposition of apatite, such as fluorapatite, treatment of lumen in teeth, treatment of caries, treatment of inflammations, for plaque reduction, for tooth bleaching, for administration of pharmacologically active substances, for individualised medication, for remineralization of teeth bleached by oxidation. Also preferable is the use of an application system or kit for depositing apatite, in particular fluorapatite, on surfaces at a layer thickness of at least partly more than or equal to 1 μm, preferably more than or equal to 2 μm, within 16 hours, in particular at body temperature in the oral environment or at 37° C. and 95% humidity.

Also a subject matter of the invention is the use of an application system or kit containing water-soluble phosphate or phosphates that can be hydrolyzed to form water-soluble phosphate ions, water-soluble fluorides or a compound releasing fluorides, and water-soluble calcium ions or compounds releasing calcium ions for a remineralizing treatment of teeth by means of once to multiple a daily, in particular once or twice daily oral application of the application system before going to bed and use of the application system over-night or during the day to form crystalline apatite deposits, in particular having a layer thickness of, in part, more than or equal to 2 μm.

Another subject matter of the invention is the use of the cross-linker solutions, in particular for use in the production, each independently, of a shaped body, comprising a cross-linker solution (a) comprising a phosphate mixture produced by mixing (i) 0.05 to 4 mol/l, 0.5 to 1.5 mol/l water-soluble phosphates or phosphates that can be hydrolyzed to form water-soluble phosphate ions, (ii) a corresponding amount of water or of a mixture of water and an organic solvent, (iii), optionally, at least one carboxylic acid and/or a buffer system, (iv) 0 to 6,000 ppm by weight water-soluble fluoride or compound releasing fluoride, and/or a cross-linker solution (b) comprising a calcium mixture produced by mixing (i) 0.1 to 2 mol/l calcium ions or compounds releasing calcium ions, (ii) a corresponding amount of water or of a mixture of water and an organic solvent, (iii), optionally, at least one carboxylic acid and/or a buffer system, and (a) and/or (b) mixing with a defined amount of a solution containing a di- or polyfunctional cross-linker comprising dialdehydes, polyepoxides, polyisocyanates, with the cross-linker, in particular, being glutardialdehyde. The phosphate solution can be used as a cross-linker solution, e.g. upon adding the cross-linker, for example glutardialdehyde. Likewise, the calcium solution can be used as a cross-linker solution, e.g. upon adding the cross-linker, for example glutardialdehyde.

For this purpose, the cross-linker is present in a cross-linker solution and is contacted to the gel thus formed. Solutions containing 0.005 to 90% by weight cross-linker in solvent with respect to the total composition, in particular in water or water-containing solvent are preferred, whereby 0.005 to 5% by weight are preferred, 0.1 to 4% by weight are particularly preferred, and about 0.1 to 1% by weight are advantageous. It is preferred to use an aqueous glutardialdehyde solution as cross-linker solution. The cross-linker solution is preferably prepared by adding the cross-linker to the phosphate solution. The preferred time of action is 1 to 200 seconds, particularly preferably 10 to 60 seconds (s). Preferably, the treatment takes approx. 20 seconds. The preferred pH value of the cross-linker solution is between 4.0 and 12.0. Final rinsing with phosphate and/or calcium solution is feasible. Also a subject matter of the invention is a cross-linker solution comprising a phosphate solution or a calcium solution and a content of cross-linker.

Moreover, the kit preferably comprises a formulation in the form (a) of an aqueous pre-treatment solution, which is synonymous to mouthwash, comprising water, 0.1 to 30% by weight of a calcium salt that dissolves well in water, in particular 5 to 15% by weight with respect to the total composition, preferably calcium lactate, calcium-chloride, calcium gluconate, calcium lacto-gluconate, a hydrate of the salts or a mixture containing two of the salts, optionally a content of a buffer system, optionally masking agent or flavouring agent and comprising a pH value of 5.0 to 12.0, or the formulation in the form of b) comprises at least one calcium salt that dissolves well in water, preferably comprising calcium lactate, calcium chloride, calcium gluconate, calcium lacto-gluconate, a hydrate of the salts or a mixture containing two of the salts, optionally a content of a buffer system, optionally a content of masking agent or flavouring agent, and common formulation excipients, such as releasing agents, HPMC, etc., in particular in the form of a water-soluble granulated material, water-soluble pellet, tablets, as sachet, powder and/or granulated material in a sachet or soluble capsules. Also a subject matter of the invention is the use of a single formulation of the form of b) in combination with a kit comprising the formulations according to the invention. The afore-mentioned mouthwash is a pre-treatment solution for pre-treating single or all teeth or a formulation from which a corresponding solution can be produced by adding water.

The composition of the kit is described in the following. The mouthwash or pre-treatment solution is composed of 0.1% by weight to 30% by weight of a calcium salt that dissolves well in water, preferably 5% by weight to 15% by weight calcium chloride or calcium lactate or calcium gluconate or calcium lacto-gluconate or other sufficiently soluble calcium salts. The solution is adjusted to a pH value between 5.0 and 12.0, preferably 8.0 to 10.0, using a suitable buffer. To improve the taste, flavouring or complexing of bad tasting compounds is feasible as long as this does not have a detrimental effect on the deposition of apatite. Suitable as buffers are all buffers showing good buffering capacity in said pH range, e.g. EDTA, Tris, HEPES or barbital-acetate buffer, but other buffer systems as well, with Tris being preferred.

Moreover, it is preferred that the formulations or mouthwashes comprise at least one buffer system, preferably a buffer substance from the group of primary alkali citrates, secondary alkali citrates and/or a salt of carboxylic acids, in particular having 1 to 20 C atoms, preferably a salt of at least one fruit acid, a salt of an alpha-hydroxy acid and/or a salt of a fatty acid, in particular having 1 to 20 C atoms. Particularly preferred salts of the aforementioned acids are the alkali, alkaline earth and/or zinc salts of citric acid, malic acid, tartaric acid and/or lactic acid or Tris. Particularly preferred salts comprise the cations of sodium, potassium, magnesium and/or zinc of the aforementioned carboxylates.

Preferably, the formulations according to the invention can be used for treatment of sensitive teeth, sensitive dental necks, acid-eroded teeth, cracked teeth, surface-abraded teeth, exposed dental necks, bleached teeth, teeth after treatment of carious tooth regions once daily (once-a-day) to several times a day, in particular once or twice a day (for example, one-day-mineralization) in order to form an apatite layer, which preferably is homogeneous and which essentially is crystalline, of 2 to more than or equal to 5 μm in thickness on the treated surfaces. As a matter of principle, the formulation can be used more frequently according to need, for example according to defined intervals.

The invention is illustrated in more detail based on the following examples and figures without limiting the invention to the examples given.

### EXAMPLE 1

Pre-treatment solution, phosphate component, and calcium component are needed for a remineralization kit. The production of the pre-treatment solution is described in the following. For component A containing phosphate ions, a solution containing 29.5 g NaH₂PO₄, 33 g Olaflur, and 27.0 g lactic acid was produced. The pH value was adjusted to 5.4 with 5 N sodium hydroxide solution and the solution was topped up to 250 ml with de-ionised water. A total of 24 ml of the solution and 6 g glycerol and 10 g of 300 Bloom pork rind gelatine were processed while heating to form a viscous gel (malleable matrix). The gel was introduced generously into the tool shown in FIG. 1a, 1b and pressed at a pressure of 0.5 bar. After cooling, the tool was opened and the phosphate component (further shaped body) was taken out. After rinsing for 40 s in an 0.5% glutardialdehyde solution, the solubility of the gel is reduced to a level such that it does not liquefy in the oral environment for up to 8 hours.

For component B containing calcium ions, a calcium chloride solution containing 29.4 g calcium chloride dihydrate and 6.3 g lactic acid was prepared. The pH value was adjusted to 5.7 with 5 N sodium hydroxide solution. The solution was topped up to 200 ml with de-ionised water. In order to produce the gel, 21.6 g of the solution and 8.24 g glycerol and 8 g calcium sulfate and 13.6 g 300 Bloom gelatine were mixed and heated (malleable matrix) and introduced into the negative mould of the jaw model (FIG. 1a, 1b). Subsequently, the tool was closed by applying pressure on the other side and chilled for 30 min at −18° C. The chilling effects a stabilisation of the otherwise very soft gels in the three-dimensional mould and reduces the tackiness. The tool can then be opened and the first shaped body comprising the Ca matrix can be taken out. After rinsing for 40 s in an 0.5% glutardialdehyde solution, the solubility of the gel is reduced to a level such that it does not liquefy in the oral environment for up to 8 hours.

FIG. 1a, 1 b: CAD model of the tool for producing the two mineralization components in the form of a splint (first or further shaped body).

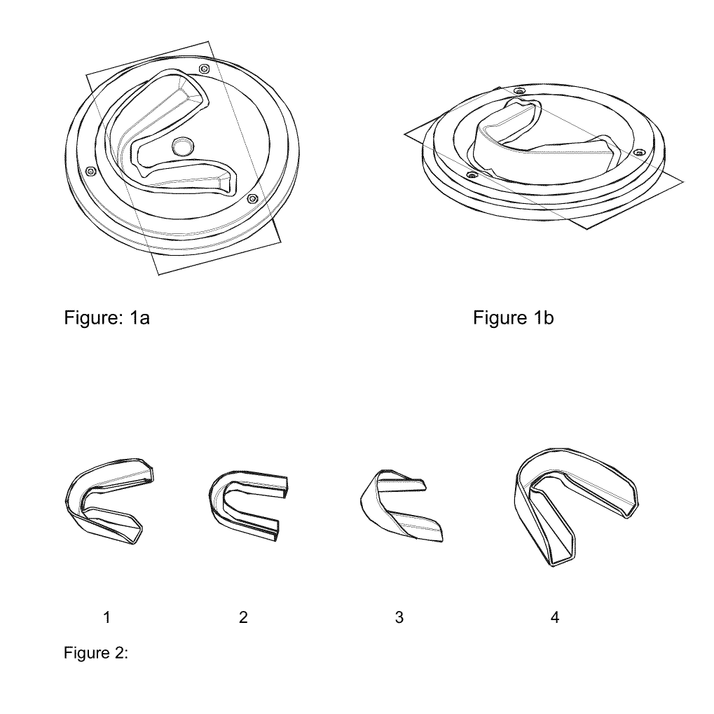

FIG. 2: The three components: 1. Protective splint (application dental splint), 2. Calcium component (first shaped body), 3. Phosphate component (further shaped body). 1 and 2 can be combined into one mould (application dental splint and the first shaped body are a unit), 3 (further shaped body) must only be added until shortly before the start of mineralization.

### EXAMPLE 2

The production of the gel splints corresponds to the description in example 1, except that the calcium component is produced together with a splint that can be deformed by thermal means, and subsequently is taken out together with said splint. This reduces the system by one component.

FIG. 2: The calcium component (4) is brought into a shape such that it is already integrated into a splint, i.e. application dental splint and the first shaped body are a unit.

For component B containing calcium ions, a calcium chloride solution containing 29.4 g calcium chloride dihydrate and 6.3 g lactic acid was prepared. The pH value was adjusted to 4.0 with 5 N sodium hydroxide solution. The solution was topped up to 200 ml with de-ionised water. A cross-linker solution was prepared for the Ca component. For this purpose, 2 g of 25% by weight glutardialdehyde solution (GDA) were topped up to 100 g with the Ca solution prepared before. For cross-linking, the shaped bodies were exposed to the cross-linker solution for 40 s. Subsequently, they were rinsed with the Ca solution containing no cross-linker component, and blown dry.

For the pre-treatment solution (mouthwash), 0.1 mol Tris buffer was added to a 1 molar calcium chloride solution and the pH was adjusted to 9.0.

Cross-linking solution for phosphate: For production of the P solution of the P component, 5.9 g Na₂HPO₄, 9.1 g lactic acid, 6.6 g Olaflur, and 0.6 g 5 M NaOH were topped up to 50 ml with de-ionised water. Subsequently, an 0.375% glutardialdehyde (GDA) solution was prepared. The further shaped body can be treated for approx. 30 sec.

### EXAMPLE 3

### Deposition of Fluorapatite

For component A containing phosphate ions, a solution containing 29.5 g NaH₂PO₄, 33 g Olaflur, and 27.0 g lactic acid was produced. The pH value was adjusted to 5.4 with 5 N sodium hydroxide solution and the solution was topped up to 250 ml with de-ionised water. A total of 24 ml of the solution and 6 g glycerol and 10 g of 300 Bloom pork rind gelatine were processed while heating to form a viscous solution. A small amount of liquid was placed in a template with a wall thickness of 500 μm and exposed to 2 bar of pressure. After solidification, the strips were removed from the template and cut into 1×1 cm squares.

For component B containing calcium ions, a calcium chloride solution containing 29.4 g calcium chloride dihydrate and 6.3 g lactic acid was prepared. The pH value was adjusted to 4.0 with 5 N sodium hydroxide solution. The solution was topped up to 200 ml with de-ionised water. In order to produce the gel, 21.6 g of the solution and 8.24 g glycerol and 8 g calcium sulfate and 13.6 g 300 Bloom gelatine were mixed and heated. The liquid gel was then spread with a squeegee to a thickness of 1 mm or pressed in a template with a wall thickness of 1 mm. After solidification, the strips were cut into 1×1 cm squares.

Pre-treatment solution (mouthwash): 0.1 mol Tris buffer was added to a 1 molar calcium chloride solution and the pH was adjusted to 9.0.

For assessment of the mineralization activity, 6 tooth discs each were etched for 10 s with 1 M HCl, rinsed with the pre-treatment solution, and covered with one piece of phosphate gel and one piece of calcium gel each. In order to make the morphological change of the tooth surface more obvious, one half of a disc was taped over first such that only half of the disc can remineralize. The samples were stored in an air-conditioned cabinet at 37° C. and 95% humidity and cleaned after 8 to 16 hours with lukewarm water and a soft toothbrush. After just one treatment, most of the tooth surface is coated by a firmly adhering layer. The layer can be up to 2.5 μm in thickness. The gels were spread over the samples after 8 to 16 hours.

### EXAMPLE 4a

For production of the P solution of the P component, 5.9 g Na₂HPO₄, 9.1 g lactic acid, 6.6 g Olaflur, and 0.6 g 5 M NaOH were topped up to 50 ml with de-ionised water. The gel was produced as described in example 1. The calcium solution for the Ca component was produced by dissolving 14.7 g CaCl₂, 3.15 g lactic acid, 10 g 5 M sodium hydroxide solution in de-ionised water to produce a total of 100 ml of the solution. The gel was produced as described in example 1. The same applies to the pre-treatment solution.

For assessment of the mineralization activity, 6 tooth discs each were etched for 10 s with 1 M HCl, rinsed with the pre-treatment solution, and then covered with one piece of phosphate gel and one piece of calcium gel each. In order to make the morphological change of the tooth surface more obvious, one half of a disc was taped over first such that only half of the disc can remineralize. The samples were stored in an air-conditioned cabinet at 37° C. and 95% humidity and cleaned after 8 to 12 hours with lukewarm water and a soft toothbrush. After just one treatment, most of the tooth surface is coated by a firmly adhering layer. In order to render the regions of successful coating versus exposed dentine more obvious to the naked eye, the samples were cleaned and then soaked in 0.1% rhodamine solution and rinsed briefly. A colorimeter was used to determine the colour difference delta E between the coated and the uncoated side. The average value is 52 (STAB 12).

### EXAMPLE 4b

The production of the kit components corresponds to example 4a, except for the P gel being treated for 30 s with an 0.375% GDA solution produced by mixing the P solution with the appropriate amount of GDA. The gel strips were then only dabbed to dry them. The Ca gels were treated with a 0.25% GDA solution for 30 s and then dabbed dry. The mineralization activity was assessed as in example 4a. The average value of delta E was 63 (STAB 5, error specification).

### EXAMPLE 5

The gels were produced as in example 4a. However, the cross-linking proceeded for 2×20 s from both sides with both gels. In this context, the GDA concentration of the Ca cross-linker solution was 0.5%. The mineralization activity was assessed as in the previous examples. A largely homogeneous layer of small needle-like crystals was seen in the electron microscope.

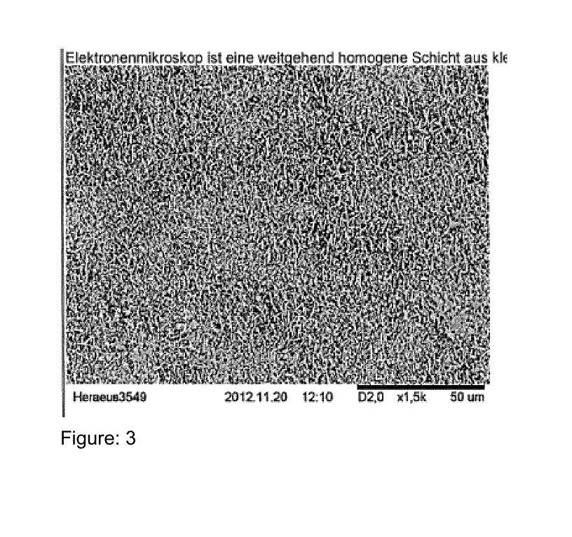

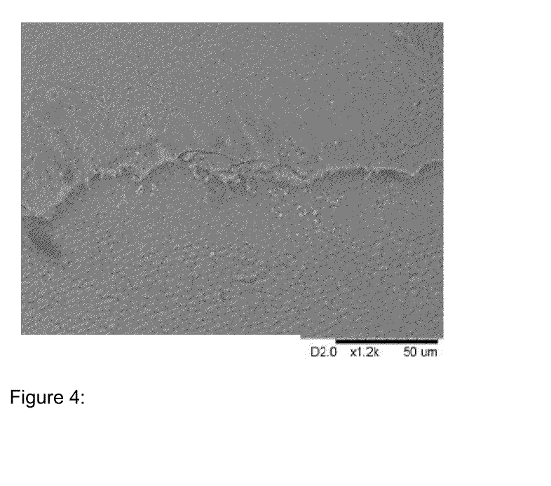

FIG. 3: Dentine surface showing growth. The pore-rich dentine surface is covered by a growth of a homogeneous layer of needle-like crystals. After treatment with the mineralization kit (GDA cross-linking on both sides).

1. A shaped body comprising a partially elastic, arch-shaped, and three-dimensional shaped body, whereby the shaped body contains a matrix containing of gel and is selected from a) a first three-dimensional shaped body containing a matrix comprising at least one gel, and
b) a further three-dimensional shaped body containing a second matrix comprising at least one gel, and at least one matrix each independently contains at least one active substance, one pharmacologically tolerable salt, solvat of an active substance or a system having a mineralizing effect.

2. The shaped body according to claim 1 wherein the shaped body is arch-shaped and comprises a groove provided to accommodate at least one tooth or at least a part of a dental arch or the shaped body is present in the form of a dental splint or the shaped body corresponds, at least in part, to a negative image of a dental arch or the shaped body adapts in form-fitting or perfectly fitting manner to at least one tooth or dental arch or corresponds, at least in part, to a negative image of a dental arch or is situated at a distance from a dental arch.

3. The Shaped body according to claim 1 wherein
a) the gel of the further shaped body comprises water-soluble phosphates or hydrolysable phosphates that form water-soluble phosphate ions and comprises a pH value of 2 to 8, and b) the gel of the first shaped body comprises water-soluble calcium ions or compounds releasing calcium ions and comprises a pH value of 3.5 to 14, and
a) the gel of the first shaped body and/or
b) the gel of the further shaped body comprise (i) a content of water or of a mixture of water and an organic solvent, and (ii), optionally, at least one carboxylic acid and/or a buffer system.

4. A formulation according to claim 1 wherein the gel of the further shaped body comprises at least one water-soluble fluoride or one compound releasing fluorides.

5. The shaped body according to claim 1 wherein the first and/or further shaped body each independently comprise, at least in part, at least one plane arranged on the outer surface or an external envelope, and the plane or envelope comprise reduced solubility with respect to aqueous media as compared to the matrix.

6. The shaped body according to claim 1 wherein the at least one gel is based on an organic gel-forming agent (a) comprising denatured collagen, hydrocolloids, hydrogels, polypeptides, protein hydrolysis products, synthetic polyamino acids, polysaccharides, polyacrylates, polyurethanes, casein, starch flour, cellulose, HPMC, gum arabic, galactomannanes, guar gum, konjac, xanthane, calcium alginate, dextrane, scleroglucan, pectin, carrageenan (κ-, ι- and λ carrageen), Agar-Agar, alginate, alginic acid, sodium alginate, calcium alginate, tragacanth or mixtures thereof,
or inorganic gel-forming agent (b) comprising bentonites, silicic acid gels or mixtures containing an inorganic gel-forming agent, (c) silicone elastomers, or (d) pharmaceutical matrix-forming agents, celluloses, acrylates, as well as mixtures of gel-forming agents (a), (b), (c) and/or (d).

7. An application system for treating teeth of vertebrates comprising at least one application dental splint, having at least one shaped body according to claim 1 wherein, the application dental splint is suitable to accommodate at least one first three-dimensional shaped body that is adapted to the splint and contains a matrix, wherein the first shaped body is adapted to insert into it at least one further three-dimensional shaped body that can be inserted into the first shaped body, whereby the further shaped body contains a second matrix.

8. The application system according to claim 7,
wherein, in an alternative embodiment
(i) a) an application dental splint accommodates a first three-dimensional shaped body containing a matrix, and b) the first three-dimensional shaped body accommodates a further shaped body containing a matrix, optionally separated by a membrane layer,
(ii) a) the application dental splint and the first shaped body are a unit, and b) the first three-dimensional shaped body accommodates a further shaped body containing a matrix, optionally separated by a membrane layer, or
(iii) a) the application dental splint comprises a matrix and accommodates a first three-dimensional shaped body containing a matrix, optionally separated by a membrane layer.

9. The application system according to claim 7 wherein (i) the further shaped body contains a matrix and the shaped body is at least partially elastic, whereby the (a) matrix comprises
(a1) at least one mineralization matrix comprising at least one gel, comprising
(a2) at least one water-soluble phosphate or hydrolysable phosphates that form water-soluble phosphate ions, and
(a3), optionally, at least one carboxylic acid and/or a buffer system
(a4) optionally, water-soluble fluorides or a compound releasing fluorides
(a5), optionally, a content of water or of a mixture of water and an organic solvent,
(ii) the first shaped body contains a matrix, and shaped body is at least partially elastic whereby the (b) matrix comprises
(b1) at least one mineralization matrix comprising at least one gel, comprising
(b2) water-soluble calcium ions or compounds releasing calcium ions, and
(b3), optionally, at least one carboxylic acid and/or a buffer system
(b4), optionally, water or of a mixture of water and an organic solvent, or
the first shaped body comprises matrices and is at least partially elastic, whereby the (c) matrices comprise
(c) partially elastic shaped body C comprises
(c1) at least one mineralization matrix comprising at least one gel, containing
(c1.1) at least one water-soluble phosphate or hydrolysable phosphates that form water-soluble phosphate ions, and
(c1.2), optionally, at least one carboxylic acid and/or a buffer system
(c1.3) optionally, water-soluble fluorides or a compound releasing fluorides
(c1.4), optionally, a content of water or of a mixture of water and an organic solvent
(c2) optionally, a membrane (layer), and
(c3) at least one mineralization matrix comprising at least one gel, containing
(c3.1) water-soluble calcium ions or compounds releasing calcium ions, and
(c3.2), optionally, at least one carboxylic acid and/or a buffer system
(c3.3), optionally, water or a mixture of water and an organic solvent, whereby the layer structure of the shaped body is c1 and c3 or c1, c2, and c3, whereby c3 adapts to the application dental splint and c1 is arranged on the inside, in particular in order to contact at least one tooth or a part of a dental arch during a dental application in a vertebrate.

10. The application system according to claim 7 wherein, in an alternative (i) the application dental splint comprises a first three-dimensional shaped body containing a matrix and the first shaped body and the application dental splint are a unit, whereby (b) the matrix comprises
(b1) at least one mineralization matrix comprising at least one gel,
(b2) water-soluble calcium ions or compounds releasing calcium ions, and
(b3), optionally, at least one carboxylic acid and/or a buffer system
(b4), optionally, water or a mixture of water and an organic solvent, or (ii) the first shaped body (b) comprising the mineralization matrix (b1) with (b2), (b3) and, optionally,
(b4) fits to be inserted into the application dental splint.

11. The application system according to claim 7 wherein in an alternative (i), the application dental splint comprises a first shaped body adapted to insert a further shaped body containing a matrix, whereby the further shaped body is three-dimensional, and the (a) matrix comprises (a1) at least one mineralization matrix comprising at least one gel, comprising
(a2) at least one water-soluble phosphate or hydrolysable phosphates that to form water-soluble phosphate ions, and
(a3), optionally, at least one carboxylic acid and/or a buffer system
(a4), optionally, water-soluble fluorides or a compound releasing fluorides
(a5), optionally, a content of water or of a mixture of water and an organic solvent, or (ii) the application dental splint according to claim 17, alternative (i), and the further shaped body (a) comprising the mineralization matrix (a1) with (a2), (a3), optionally (4a), and, optionally, (a5), optionally separated by a membrane, are present as a unit.

12. A method for producing the shaped body according to claim 1 wherein a mouldable matrix is introduced into a first work piece having a recess that corresponds to at least a partial negative image of a jaw or partial negative image of a dental arch or is derived from either, and is formed together with a second work piece that corresponds, at least in part, to a positive image of a jaw or partial positive image of a dental arch, at normal pressure or excess pressure, optionally is being cooled, whereby the formed matrix is taken out as a shaped body after forming, whereby the first or the further shaped body is obtained.

13. The method for producing the application system according to claim 7 wherein (a) an application dental splint (a.1) is provided or
(a.2) formed, in particular by means of injection moulding, extrusion or other method known to a person skilled in the art,
(b.1) the first shaped body is introduced into the application dental splint (b.1.1) in pre-formed manner or (b.1.2) is introduced into the application dental splint and formed by means of 2K injection moulding, or (b.1.3) the first shaped body is formed by the user and is introduced into the application dental splint.

14. The method according to claim 13, wherein
(c.1) the further shaped body is introduced into the application dental splint (c.1.1) in pre-formed manner or (c.1.2) is introduced into the first shaped body by means of 2K injection moulding, optionally separated by means of a membrane, or (c.1.3) the further shaped body is formed by the user and is introduced into the first shaped body in the application dental splint.

15. The method according to claim 12 wherein
a) the further shaped body comprising a matrix and at least one gel containing at least one water-soluble phosphate or compound releasing phosphate, gelatine and glycerol, optionally at least one carboxylic acid and/or a buffer system, optionally water-soluble fluorides or a compound releasing fluorides, optionally a content of water or of a mixture of water and an organic solvent, is produced in that
gel, in particular warm, non-solidified gel, is introduced into the first work piece as mouldable matrix, is pressed under pressure, is cooled optionally, the solidified gel is taken out as matrix or as shaped body comprising the matrix, subsequently the matrix is chemically cross-linked on at least one outer plane or chemically cross-linked on the outside into an envelope, or coated and/or
b) the first shaped body comprising a matrix and at least one gel containing water-soluble calcium ions or compounds releasing calcium ions, optionally at least one carboxylic acid and/or a buffer system, optionally water or a mixture of water and an organic solvent, is produced in that
gel, in particular warm, non-solidified gel, is introduced into the second work piece as mouldable matrix, is pressed under pressure, is cooled optionally, the solidified gel is taken out as matrix or as shaped body, subsequently the matrix is chemically cross-linked on at least one external plane or chemically cross-linked on the outside into an envelope or is coated in a plane or with an envelope.

16. Kit comprising an application system comprising an application dental splint, a first shaped body and/or a further shaped body according to claim 1, as well as, optionally, a pre-rinsing solution.

17. (canceled)
